(19) United States
(12) Patent Application Publication
(10) Pub. No.: US 2013/0098365 A1
(43) Pub. Date: Apr. 25, 2013

(54) METHOD AND SYSTEM FOR PREVENTION OF SURGICAL FIRES

(71) Applicant: Michelle C. BOHNER, San Francisco, CA (US)

(72) Inventor: Michelle C. BOHNER, San Francisco, CA (US)

(21) Appl. No.: 13/280,224

(22) Filed: Oct. 24, 2011

(51) Int. Cl. A62C 3/00; A61M 16/04

(52) U.S. Cl. 128/207.15

(57) ABSTRACT

System and method for prevention of surgical fires inside a patient's airway. The system includes a specially adapted endotracheal tube which carries sublines (in addition to the main line carrying anesthetic gases) for carrying an air sample back from the distal end of the tube to a remote oxygen sensor. Upon the sensor sensing an undesirably or dangerously high level of oxygen within the patient's airway, the system operates alarms to alert the surgical personnel, and also operates a controllable valve to admit an inert gas into other sublines associated with the endotracheal tube and which deliver the inert fire suppressing gas to the distal end of the tube, proximal to the cuff, inside the patient's airway.

### FIELD OF THE INVENTION

This invention relates to a medical apparatus and specifically to a system for preventing surgical fires involving anesthetic gases.

### BACKGROUND

The problem of surgical or operating room fires due to the combination of anesthetic gases and an ignition source is well known, see for instance Gedebou US2006/0058784, Lampotang et al. US2006/0150970 and Foltz US7,296,571. Typically the ignition source is a heat-producing surgical instrument, such as an electrosurgery cautery device or another electrosurgery tool or a laser. Oxygen is present since it is often administered to a patient during surgery as part of the anesthesia. The combustible material is, for instance, the patient's tissue or parts of the anesthesia equipment. Such fires occur when the oxygen administered to the patient leaks into the patient's upper airway or the operating room, causing a highly oxidized environment and increased flammability of human tissue and surgical equipment. A fire can ignite when this fuel is exposed to an ignition source. The oxygen is administered in a number of ways. One is by use of a cannula which is applied to the nose. Another method is a face mask. Another method is an endotracheal tube which is inserted into the patient's mouth and down into the throat so as to administer the oxygen well down into the patient's airway. Near the distal end of such tubes there is typically a cuff which is inflated to seal the patient's airway to the outer circumference of the tube to prevent the anesthesia gases from leaking out of the patient's esophagus and into the patient's throat and/or ambient atmosphere. However, often there are gas leaks because the cuff does not properly engage with the patient's anatomy, allowing the oxygen to leak past the cuff, causing an increased risk for fire.

### SUMMARY

Therefore surgical fires are traumatic and a well known risk of surgical procedures. In addition to pure oxygen, nitrous oxide (also used in anesthesia) can act as the fire oxidizer. Much surgical equipment is made of plastic and becomes more flammable in an oxidized environment, such as when anesthesia gas leaks into the ambient atmosphere. Therefore, both the patient's tissues and much surgical equipment can serve as a fuel source. It is known that ear, nose and throat surgeries commonly lead to surgical fires due to poor ventilation of the throat and airway. In these procedures the electrosurgical device operates especially in close proximity to the plastic (e.g., PVC) endotracheal tube used to deliver the anesthetic gases, potentially resulting in damaging airway fires inside the patient's throat or mouth. This can occur when the anesthetic gases leak around the cuff provided on such tubes into the patient's upper airway.

The tube cuff designs are not particularly efficient due to variability in patient anatomy. The cuff pressures are supposed to be monitored by the anesthesiologist but this is not done as frequently as desired. There have been a number of endotracheal tubes developed to reduce gas leaks, but none are particularly effectively. Further, the above referenced patent documents do not deal especially with endotracheal tubes but instead focus on oxygen administered by face mask or nose cannula.

The present system detects and prevents surgical fires, with use of an endotracheal tube. The system includes an oxygen sensor to detect a flammable atmosphere by the level of oxygen. Upon detecting a predetermined dangerously high oxygen concentration proximal to the endotracheal tube cuff, the system triggers an automated response to reduce or eliminate the presence of the oxygen. The response includes auditory and visual warnings, such as an audio alarm, a gauge, or set of lights, such as LEDs to provide a real time display of the risk level to the surgical personnel. Further, the active response of the system includes delivery of an inert gas to the site of the anesthetic gas leak to suppress the possibility of a fire. A typical inert gas is nitrogen. The system is operated and controlled using a conventional micro-controller driven by the oxygen sensor.

### BRIEF DESCRIPTION OF DRAWINGS

FIG. 1 shows a system in accordance with the invention.

FIG. 2 shows the endotracheal tube of FIG. 1 in detail.

FIG. 3 shows a cross section of a portion of FIG. 2 along lines AA.

FIG. 4 shows detail of the controller of the FIG. 1 system.

### DETAILED DESCRIPTION

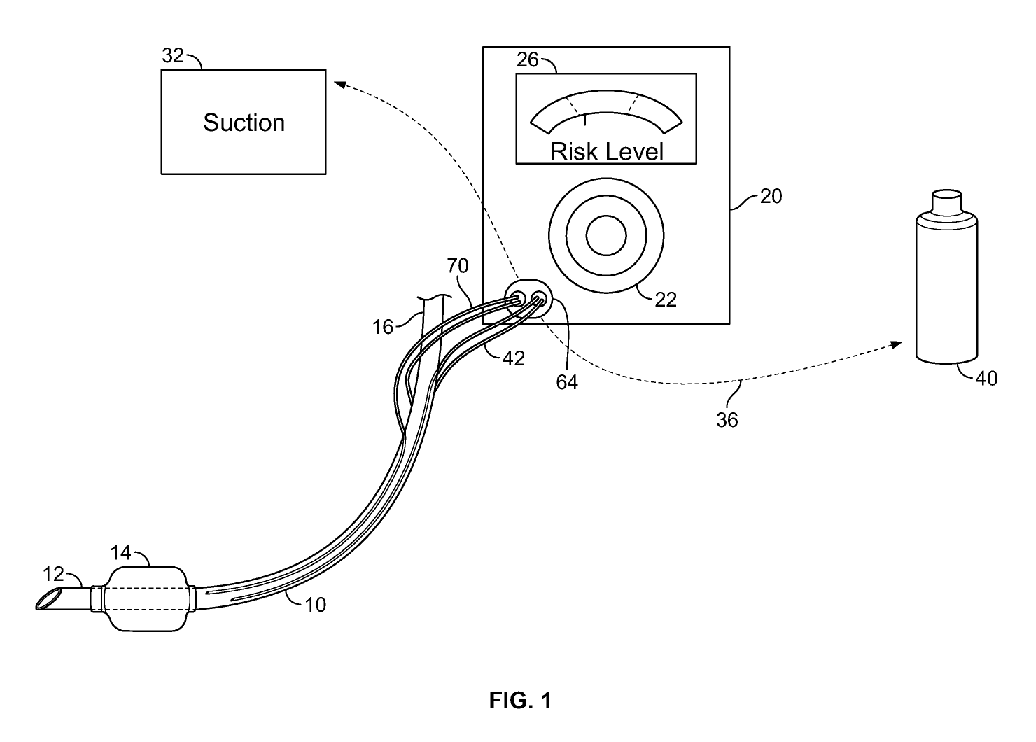

FIG. 1 shows in a block diagram a system in accordance with the present invention. FIG. 1 does not show the conventional electrosurgery device, or the usual anesthesia equipment except for the endotracheal tube 10, which in several respects here is not a standard endotracheal tube. However, this endotracheal tube 10 has distal end 12 which is inserted into the patient's throat and the surrounding cuff 14, both conventional. The proximal end 16 of tube 10 is conventionally coupled to a source of anesthetic gases, such as oxygen, nitrous oxide, etc.

Also included in the system is a controller housed in a controller housing 20 and including a driver for an audio alarm located inside the housing such as a loud speaker, not shown, and a visual indicator of the oxygen level, such as a gauge 26 or set of light-emitting diodes 22, as explained further below.

The controller housing is connected via a port (not shown) to a conventional external source of suction 32, such as a suction pump or the suction mains typically provided in an operating room. Also provided is a connection 36 to a conventional source of a fire suppressant (inert) gas 40. Source 40 may be a conventional inert gas source provided in the operating room. The internal arrangements of the controller are explained in detail below. A conventional oxygen sensor, for instance, a partial pressure oxygen sensor, of the type commercially available, is housed inside the controller housing 20 and monitors fluctuations in the concentration of oxygen on the outside circumference of the proximal end of the endotracheal tube cuff, to sample the oxygen concentration proximal to the cuff, via suction. Similar tubes deliver the inert gas to the same location. Hence although the oxygen sensor is located inside the controller housing 20, it continuously receives samples of the atmosphere inside the patient's throat at the proximal end of cuff 14 via line 42 at port 64, as explained further below. The oxygen sensor conventionally generates a voltage signal directly proportionally to ambient sensed oxygen concentration. The source of suction 32 connected to the controller housing 20 constantly pulls air through the flow-through head of the oxygen sensor from line 42. The suction source 32 is connected to the housing via a port in the back of the housing 20. A tube on the inside of the housing connects to this source 32 and couples suction to one side of the oxygen sensor flow-through head. On the other side of the flow-through head a different tube leads to the front port 64 from the inside of the housing. On the outside of the housing at the front port 64 a tube 70 is connected that leads to the endotracheal tube 10. Tube 70 branches to smaller diameter tubes that line the outside circumference of the endotracheal tube or are manufactured inside the walls of the endotracheal tube. Therefore the suction pump draws a sample of air through the oxygen sensor.

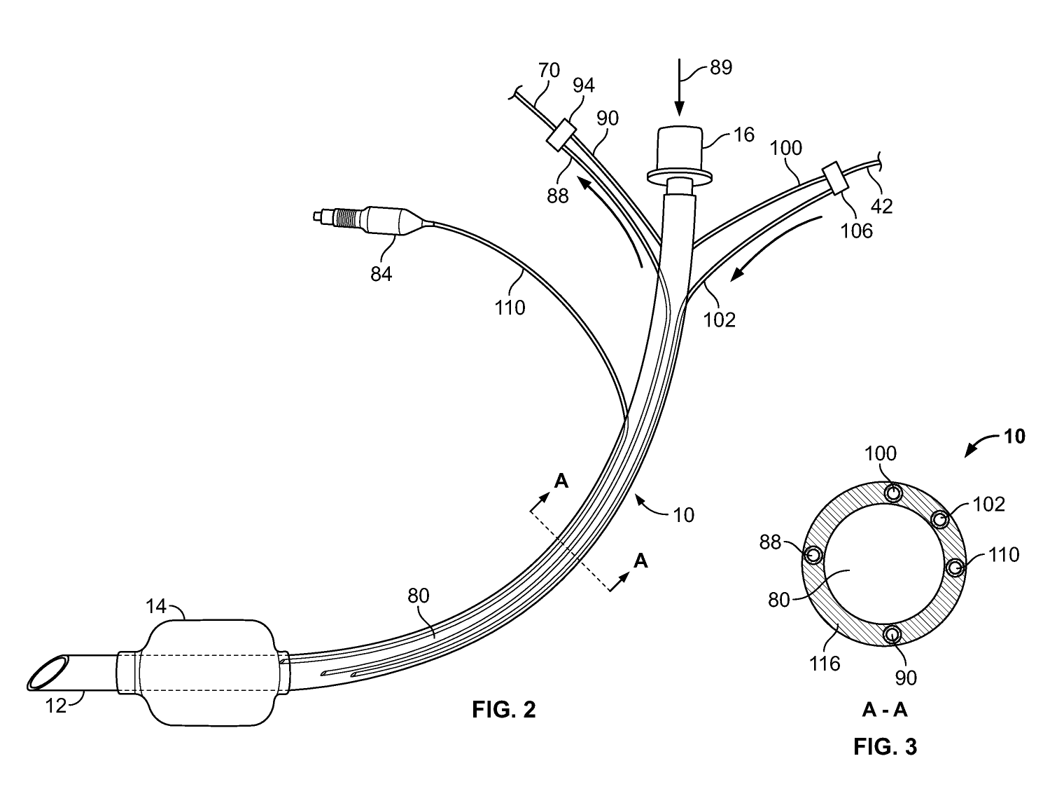

FIG. 2 shows detail of endotracheal tube 10 of FIG. 1 with similar elements carrying the same reference numbers. Tube 10 carries the anesthetic gases from their source 89 via a coupling 16, all of which are conventional. Also conventional is adapter fitting 84 to couple to an air syringe to inflate the endotracheal tube cuff 14 via line (tube) 110 which here extends along or in the wall of tube 10 and extends to the inside of the balloon cuff 14. This structure is conventional also. From the outside of port 64, tube 42 is divided at coupling 106 into smaller tubes 100 and 102. The end of these tubes 100, 102 is immediately adjacent the proximal end of the balloon cuff 14. Inert gas source 40 feeds to a port in the back of the housing. A tube inside the housing connects the gas source to the solenoid and is coupled to the front port 64. Front port 64 is connected to line 70 which branches at coupling 94 to smaller tubes 88 and 90 that run along the outside or inside the wall of the endotracheal tube. The end of these smaller tubes is immediately adjacent the proximal end of the balloon cuff. Hence port 64 couples to two separate lines, one to deliver the inert gas and the second to couple the suction.

FIG. 3 shows a cross section of the tube 10 along line A-A of FIG. 2. Central channel 80 carries the anesthetic gases. Tube 10 conventionally has a wall 116 in which are defined sampling lines (channels) 88 and 90, and fire suppression gas supply lines (channels) 100 and 102. Also defined in the tube wall 116 is cuff inflation line 110. Of course, this provision of lines or channels in the wall 116 of tube 10 is not limiting. The various lines can be provided by other means such as being independent tubes attached to the inside or outside of the wall of tube 10. Typically tube 10 is molded of plastic such as polyvinyl chloride (PVC) and is a disposable item. The actual dimensions of the various structures shown in FIG. 2 are largely conventional. The diameters of the various lines 70, 88, 90, 42, 100, 102, 110 is a matter of engineering choice, so long as sufficient airflow is provided for oxygen sampling purpose and sufficient inert gas is provided. Exemplary diameters of tubes 88, 90, 100, 102 are outside diameter 3/32″ (2.5 mm), inside diameter 1/32″ (0.8 mm). Tubes 42 and 70 have an exemplary inside diameter of ⅛″ to ¼″ (3 to 6 mm) and corresponding outer diameter. The diameters of the tubes are not critical. The number of lines (tubes) associated with tube 10 for inert gas delivery and air sampling is also a matter of engineering choice.

The structure of FIGS. 2 and 3 is a subsystem of the FIG. 1 system and may be sold separately since it is usually disposable, and typically used for only one surgical procedure, while the remainder of the FIG. 1 system is typically reused many times, for instance installed in an operating room or surgical suite.

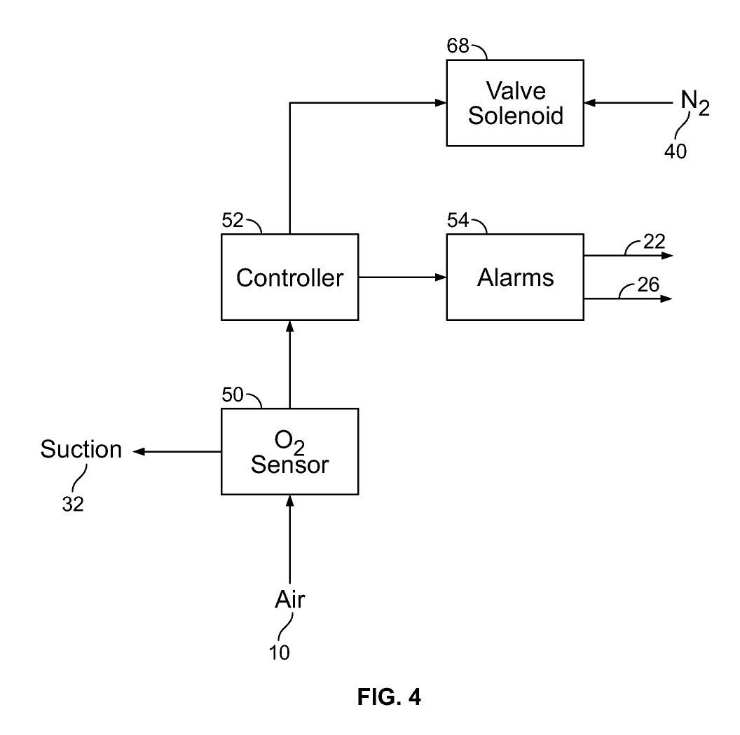

FIG. 4 shows in a block diagram the controller components housed within controller housing 20. These include the oxygen sensor 50, a micro-controller 52 typically mounted on an associated printed circuit board with the associated conventional interface components, and an alarm driver circuit 54 also mounted on the printed circuit board for driving the audio alarm and the visual alarm 22, 26, both of which are conventional. Suction from source 32 is applied to pull the sampled air through the oxygen sensor 50. This air after being sampled by oxygen sensor 50 is ventilated. Also provided, and driven by the micro-controller 52 and its interface circuitry, is a conventional solenoid valve 68 which is operated in accordance with signals sent by the micro-controller 52 to turn on gas flow from the nitrogen source 40, which is connected at the back of the housing and thereby at port 64 to line 70 of the endotracheal tube.

The micro-controller 52 (or other suitable controller) interprets the signals from oxygen sensor 50. First, the voltage signal, for instance, from 0 to 60 millivolts amplitude supplied by oxygen sensor 50, is conventionally amplified by an instrument operational amplifier to be a direct current voltage signal, for instance 0 to 5 volts amplitude. This range is specific to the oxygen sensor. This amplified voltage is interpreted by the micro-controller 52 firmware and digitally mapped to a corresponding bit value between 0 and 1,023. For instance, 0 volts equates to a 0 bit value and 5 volts equates to a 1,023 bit value. The bit values are mapped to a set of three designated Cases 0, 1 or 2 in the firmware associated with the micro-controller 52, corresponding to the atmospheric oxygen concentration, and elicit different responses. For instance, Case 0 corresponds to oxygen value 0 to 341, which is 0% to 30% oxygen. Case 1 corresponds to oxygen values 342 to 682 which is 31% to 60% oxygen. Case 2 corresponds to oxygen values 683 to 1,023, which is 61% to 100% oxygen. The corresponding oxygen concentrations to the Case numbers can be varied depending on engineering choice.

Formulas are applied by the micro-controller firmware to calculate these values as follows:

V=(4.88×10⁻³)×Byte #

O₂=0.05×V,where V is the voltage and O₂ is the concentration of oxygen.

Hence the three Case numbers are assigned respectively to three Cases in the associated firmware which elicit appropriate responses in the alarm driver 54 and the solenoid valve 68. Writing suitable firmware would be routine in light of this disclosure.

Solenoid valve 68 thereby controls delivery of the flame retardant gas, for instance, nitrogen from source 40. The controller in one version uses a 12 volt direct current solenoid valve 60 controlled by the micro-controller 52 and powered by the same power supply (not shown) as conventionally connected to the other components of the controller. Typically solenoid value 68 is closed and then operated to be (open) only for Case 2 when the oxygen concentration exceeds 60%. Hence the controller 52 activates the solenoid 68 to release the nitrogen gas through the endotracheal tube gas delivery line 70 shown in FIG. 1. Also provided is a conventional power supply for the controller, not shown.

When subsequently the oxygen sensor 50 indicates that the ambient oxygen concentration has dropped below 60%, the solenoid value 68 is deactivated (closed) by the micro-controller 52, shutting off the supply of nitrogen gas. Thus an active feedback loop is established, effectively maintaining a safe surgical environment in terms of oxygen concentration inside the patient's airway.

In one embodiment nitrogen is used as the flame retardant gas because it is a natural component of atmospheric air, readily available in most operating rooms, and cost effective. Also of course, it is compatible with patient health, unlike, for instance, high concentrations of carbon dioxide. But other inert gases may be used as a substitute for nitrogen.

The same three cases which control the solenoid valve also control the alarm driver 54. For Case 1 which is the sensed oxygen concentration below 30%, the low risk response Case 0 is activated, thus illuminating, for instance, a green LED 22 in the visual display of FIG. 1. Typically no audio alarm is provided at this point. When the sensed oxygen concentration is at 31%-60%, the moderate risk response which is Case 1 is activated so that, for instance, a yellow LED in the visual display 22 is activated. When the sensed oxygen concentration is detected at the danger level of above 60%, the high risk response Case 2 is activated at which point the loud speaker is activated to sound a buzzer or other type suitable audio alarm and the red LED in the visual display 22 is activated. Of course, any other type of alarms can also be provided. The LEDS are in addition to the oxygen gauge indicator 26 which provides a numeric read out.

Various types of oxygen sensors may be used, for instance, a partial pressure oxygen sensor supplied by Apogee has been found suitable. Also suitable is a zirconium dioxide oxygen sensor or galvanic oxygen sensor.

It has been found that using such a system, when the oxygen is sensed to be at the danger level, its concentration inside the patient's airway can be reduced to a normal or fire safe level in as little as 20 or 30 seconds. Moreover, the determination of the 60% oxygen level as the danger level, while not limiting, has been found by experiment to be a typical level above which tissue ignition will take place and below which tissue ignition is not likely to take place. PVC ignition will take place at lower oxygen concentration, e.g. 21% and this may be used as a critical level in addition or in the alternative.

This disclosure is illustrative and not limiting. Further modifications will be apparent to those skilled in the art in light of its disclosure and are intended to fall within the scope of the appended claims.

We claim:

1. A system comprising:
an endotracheal tube defining a channel adapted to carry anesthetic gas from its proximate end to its distal end, and having a cuff near its distal end;
wherein the tube includes at least one channel adapted to apply suction at the proximal end of the cuff and extending to the proximal end of the tube; and at least one channel adapted to carry an inert gas to the proximal end of the cuff and extending to the proximal end of the tube;
an oxygen sensor and a source of suction coupled to the proximal end of the suction carrying channel;
a controllable valve coupling a source of inert gas to the proximal end of the inert gas carrying channel; and
a controller coupled to receive a signal from the oxygen sensor and coupled to operate the controllable valve responsive to the signal.

2. The system of claim 1, wherein the controllable valve includes a solenoid.

3. The system of claim 1, further comprising a visual or audio oxygen level indicator coupled to the controller.

4. The system of claim 1, wherein the suction and inert gas channels are each defined in a wall of the tube.

5. The system of claim 1, further comprising:
at least one additional channel adapted to apply suction and defined in the tube; and
a manifold near the proximal end of the tube and which couples together the suction channels.

6. The system of claim 1, further comprising:
at least one additional channel adapted to carry the inert gas and defined in the tube; and
a manifold near the proximate end of the tube and which couples together the inert gas channels.

7. The system of claim 1, further comprising a housing, wherein the sensor, controller, controllable valve, source of inert gas and a source of suction are mounted to the housing, and the housing having ports adapted to connect respectively to the suction and inert gas channels near the proximal end of the tube.

8. The system of claim 1, further comprising a port on the tube coupled to the cuff, to inflate the cuff.

9. The system of claim 1, wherein the oxygen sensor is a partial sensor pressure.

10. The system of claim 1, wherein the controller operates the valve when the signal indicates an oxygen level greater than 60%.

11. An article of manufacture comprising:
an endotracheal tube defining a channel adapted to carry anesthetic gas from its proximate end to its distal end, and having an inflatable cuff near the distal end;
wherein the tube includes at least one channel adapted to apply suction at the proximal end of the cuff, and extending to the proximal end of the tube; and at least one channel adapted to carry an inert gas to the proximal end of the cuff, and extending to the proximal end of the tube.

12. The article of claim 11, wherein the suction and inert gas channels are each defined in a wall of the tube.

13. The article of claim 11, further comprising:
at least one additional channel adapted to apply suction and defined in the tube; and
a coupling near the proximal end of the tube and which couples together the suction channels.

14. The article of claim 11, further comprising:
at least one additional channel adapted to carry the inert gas and defined in the tube; and
a coupling near the proximal end of the tube and which couples together the inert gas channels.

15. The article of claim 11, further comprising a port on the tube coupled to the cuff, to inflate the cuff.

16. A method of preventing or suppressing surgical fires, comprising the acts of:
inserting in a patient an endotracheal tube defining a channel adapted to carry anesthetic gas from its proximate end to its distal end, and having a cuff near the distal end;
wherein the tube includes at least one channel adapted to apply suction at the proximal end of the cuff, and extending to the proximal end of the tube; and at least one channel adapted to carry an inert gas to the proximal end of the cuff, and extending to the proximal end of the tube;
sensing a level of oxygen at a proximal end of the suction channel while applying suction at the proximal end of the cuff by the suction channel;
receiving a signal indicating the sensed level of oxygen; and
providing inert gas to a proximal end of the inert gas channel via a controllable valve, responsive to the signal.
